(19) United States
(12) Patent Application Publication
(10) Pub. No.: US 2013/0202686 A1
(43) Pub. Date: Aug. 8, 2013

(54) LIPOSOME COMPOSITION AND PROCESS FOR PRODUCTION THEREOF

(71) Applicant: TERUMO KABUSHIKI KAISHA, Shibuya-ku (JP)

(72) Inventors: Keiko YAMASHITA, Ashigarakami-gun (JP); Shigenori Nozawa, Ashigarakami-gun (JP)

(73) Assignee: TERUMO KABUSHIKI KAISHA, Shibuya-ku (JP)

(21) Appl. No.: 13/837,633

(22) Filed: Mar. 15, 2013

Related U.S. Application Data
(63) Continuation of application No. PCT/JP2011/080304, filed on Dec. 27, 2011.

(30) Foreign Application Priority Data
Dec. 27, 2010 (JP) 2010-291110

(51) Int. Cl. A61K 9/00; A61K 9/127

(52) U.S. Cl. CPC A61K 9/0002; A61K 9/1278; A61K 9/127
USPC 424/450

(57) ABSTRACT

A liposome composition into which a drug can be introduced in a high encapsulation amount, which has sustained release properties to such an extent that an effective concentration can be maintained at a clinically satisfactory level, and which is suitable for subcutaneous administration or the like. The liposome composition includes: a first liposome which has an outer membrane composed of a multilayered lipid bilayer; and a plurality of second liposomes which are accommodated in a first liposome inner region defined by the outer membrane and each of which has an outer membrane composed of a lipid bilayer. The lipid bilayer of the second liposomes can be multilayered. The liposome composition has second liposome inner regions each defined by the outer membrane of each of the second liposomes. An ion gradient is formed at least between each of the second liposome inner regions and the outside of the first liposome.

### RELATED APPLICATION(S)

This application claims priority as a continuation application under 35 U.S.C. §120 to PCT/JP2011/080304, which was filed as an International Application on Dec. 27, 2011 designating the U.S., and which claims priority to Japanese Application No. 2010-291110 filed in Japan on Dec. 27, 2010. The entire contents of these applications are hereby incorporated by reference in their entireties.

### TECHNICAL FIELD

Disclosed is a sustained-release liposome composition containing an effective component such as a drug.

### BACKGROUND DISCUSSION

Drugs needing frequent administration have a problem that frequent hospitalization and pain due to a puncture or the like can impose heavy burdens on the patient. In addition, it is difficult for patients suffering from difficulty in swallowing to take a drug by mouth, so that an administering method other than peroral administration is desirable. Also, for patients needing a care giver such as patients of dementia, brain diseases and Parkinson's disease, it is difficult to take the drug under the patients' own control. In these cases, therefore, an administering method other than peroral administration or a treating method not needing frequent administration is desirable. Further, for patients whose daily living is hindered as soon as the drug stops working, such as patients of autonomic imbalance, a therapeutic method is desirable in which the drug's efficacy is not lost in a short time but can continue for a long period of time. Or, as for the pain after an operation, the patient may experience unbearable pain as soon as the drug stops working, which may influence the rehabilitation and may cause a delay in leaving the hospital. Therefore, if the drug can maintain its efficacy and suppress pain, for example, for five to seven days after an operation, it is considered that the postoperative rehabilitation can be promoted, which can in turn contribute to leaving the hospital earlier.

Sustained release preparations by which a drug's efficacy can be maintained for a long time can provide means for enhancing patients' QOL in all disease regions.

Many of the sustained release preparations which have been investigated heretofore are microspheres based on the use of polylactic acid-glycolic acid copolymer (PLGA). For instance, as disclosed in Biomaterials, 28 (2007), 1882-1888, PLGA microspheres using donepezil hydrochloride, an effective ingredient of Aricept (registered trademark; Eisai Co., Ltd.) which can be used as an Alzheimer-type dementia treating agent, have been investigated and sustained release properties have been obtained therewith. In the case of using PLGA, however, it is difficult to encapsulate, for example, a water-soluble drug in high concentration and with high efficiency, and there are problems yet to be solved in order to attain a high drug encapsulation amount. In addition, the use of PLGA has a problem in that the use of an organic solvent in the preparation process can make the removal of the organic solvent indispensable. See, for example, JP-T-2001-505224 and JP-T-2001-522870. Local intensification of acid upon decomposition of PLGA can cause inflammation.

Other than the above, some approaches of sustained release preparations based on the use of a local anesthetic such as bupivacaine have also been investigated, but they still have a problem achieving retention properties that can suppress pain tending to last for five to seven days after an operation. See, for example, Anesthesiology, 101 (2004), 133-137. Multivesicular liposome (MVL) has been developed as a lipid-based sustained release drug support for local or systemic drug delivery. See, for example, JP-T-2001-505224 and JP-T-2001-522870. This approach, however, is also not yet satisfactory in regard to drug encapsulation amount and sustained release time.

### SUMMARY

According to an exemplary aspect, disclosed is a liposome composition, comprising: a first liposome having an outer membrane comprised of a multilayered lipid bilayer; and a plurality of second liposomes accommodated in a first liposome inner region defined by the outer membrane of the first liposome, the second liposomes each having an outer membrane comprised of a lipid bilayer, wherein the liposome composition has second liposome inner regions each defined by the outer membrane of each of the second liposomes, and an ion gradient is formed at least between each of the second liposome inner regions and the outside of the first liposome. The lipid bilayer of the second liposomes can be multilayered.

According to an exemplary aspect, disclosed is a process for producing a liposome composition provided with an ion gradient between the inside and the outside of an outer membrane, the process comprising: mixing a first inner aqueous phase solution containing a compound for forming the ion gradient with a lipid-containing water-miscible solvent in a volume ratio from 0.7 to 2.5 so as to prepare a first emulsion; mixing a second inner aqueous phase solution with the first emulsion in a volume ratio of not less than 0.7 so as to prepare a second emulsion; and replacing an outer aqueous phase of the second emulsion with an aqueous solution which is lower than the first inner aqueous phase solution in the concentration of the compound for forming the ion gradient.

### BRIEF DESCRIPTION OF DRAWINGS

FIG. 1 is a photograph (magnification: 32,000) obtained by transmission electron microscope (TEM) observation of a section of a liposome composition, after introduction of a drug, produced in Preparation Example 2, according to an exemplary embodiment.

FIG. 2 is a graph representing the results of pharmacokinetics profile of donepezil liposome (Comparative Example 2) prepared by Extrusion Method-1, according to an exemplary embodiment.

FIG. 3 is a graph representing the results of pharmacokinetics profile of liposome compositions obtained in Preparation Examples 2, 3 and 4, according to an exemplary embodiment.

FIG. 4 is a graph representing the results of pharmacokinetics profile of liposome compositions obtained in Preparation Examples 5 and 6, according to an exemplary embodiment.

FIG. 5 is a graph representing the results of pharmacokinetics profile of a liposome composition prepared in Preparation Example 12, according to an exemplary embodiment.

### DETAILED DESCRIPTION

According to an exemplary aspect, disclosed is a liposome composition in which a drug is moved from the outside into the inside along an ion gradient, into which the drug can thereby be introduced in a high encapsulation amount with high efficiency, and which has sustained release properties to such an extent that an effective concentration can be maintained at a clinically satisfactory level. According to an exemplary aspect, disclosed is a process for production of the liposome composition.

Disclosed are the following exemplary aspects.

(1) A liposome composition including: a first liposome having an outer membrane comprised of a multilayered lipid bilayer; and a plurality of second liposomes accommodated in a first liposome inner region defined by the outer membrane, the second liposomes each having an outer membrane comprised of a lipid bilayer, in which the liposome composition has second liposome inner regions each defined by the outer membrane of each of the second liposomes, and an ion gradient is formed at least between each of the second liposome inner regions and the outside of the first liposome. The lipid bilayer of the second liposomes can be multilayered.

(2) The liposome composition as described in the above paragraph (1), in which the ion gradient is a proton concentration gradient, and the pH in the second liposome inner region or the pH in the second liposome inner region and the first liposome inner region is lower than the pH in the outside of the first liposome.

(3) The liposome composition as described in the above paragraph (1) or (2), in which the first liposome has an average particle diameter within a range of 1 to 20 μm.

(4) The liposome composition as described in any of the above paragraphs (1) to (3), in which a drug is contained in the second liposome inner region or in the second liposome and first liposome inner regions.

(5) The liposome composition as described in the above paragraph (4), in which the drug is contained in a molar ratio (mol/mol) of not less than 0.05, based on total lipid.

(6) The liposome composition as described in any of the above paragraphs (1) to (5), in which lipid membranes of the first liposome and the second liposomes are each comprised of a lipid including a phospholipid and cholesterol.

(7) The liposome composition as described in the above paragraph (6), in which the phospholipid is a saturated phospholipid.

(8) A process for producing a liposome composition provided with an ion gradient between the inside and the outside of an outer membrane, the method including the steps of: mixing a first inner aqueous phase solution containing a compound for forming the ion gradient with a lipid-containing water-miscible solvent in a volume ratio of from 0.7 to 2.5 so as to prepare a first emulsion; mixing a second inner aqueous phase solution with the first emulsion in a volume ratio of not less than 0.7 so as to prepare a second emulsion; and replacing an outer aqueous phase of the second emulsion with an aqueous solution which is at least lower than the first inner aqueous phase solution in the concentration of the compound for forming the ion gradient.

(9) The process for producing a liposome composition as described in the above paragraph (8), in which the ion gradient is a proton concentration gradient.

(10) The process for producing a liposome composition as described in the above paragraph (8) or (9), in which the first inner aqueous phase solution contains a sulfate.

(11) The process for producing a liposome composition as described in the above paragraph (10), in which the sulfate is ammonium sulfate.

(12) The process for producing a liposome composition as described in any of the above paragraphs (8) to (11), further including a step of introducing a drug into the inside of the liposome composition by a driving force due to the ion gradient.

In an exemplary embodiment, provided is a liposome composition including: a first liposome having an outer membrane comprised of a multilayered lipid bilayer; and a plurality of second liposomes accommodated in a first liposome inner region defined by the outer membrane, the second liposomes each having an outer membrane comprised of a lipid bilayer, in which the liposome composition has second liposome inner regions each defined by the outer membrane of each of the second liposomes, and an ion gradient is formed at least between each of the second liposome inner regions and the outside of the first liposome. The lipid bilayer of the second liposomes can be multilayered. In an exemplary embodiment, the liposome composition permits a drug to be encapsulated therein with high efficiency and is capable of long-time sustained release of the drug.

By an exemplary process for producing a liposome composition, it is possible to obtain a liposome composition which permits a drug to be encapsulated therein with high efficiency and which is capable of long-time sustained release of the drug.

### Phospholipids

The phospholipid can be a main lipid constituting a lipid bilayer (hereinafter, sometimes also referred to simply as lipid membrane or liposome membrane) of a liposome composition according to an exemplary aspect. The phospholipid can be a main component of the lipid bilayer. For example, the phospholipid is an amphipathic substance which has both a hydrophobic group composed of a long chain alkyl group and a hydrophilic group composed of a phosphate group in its molecule. Examples of the phospholipid include: glycerophosphoric acids such as phosphatidylcholine (=lecithin), phosphatidylglycerol, phosphatidic acid, phosphatidylethanolamine, phosphatidylserine, and phosphatidylinositol; sphingophospholipids such as sphingomyelin; natural or synthetic diphosphatidylphospholipids such as cardiolipin, and their derivatives; hydrogenation products of these phospholipids such as hydrogenated soybean phosphatidylcholine (HSPC), hydrogenated egg yolk phosphatidylcholine, distearoylphosphatidylcholine, dipalmitoylphosphatidylcholine, and dimyristoylphosphatidylcholine. The phospholipids can be used either singly or in combination of a plurality of ones of them.

Other Additives than Phospholipids

The liposome composition according to an exemplary aspect may include other membrane component(s) together with the above-mentioned exemplary main component (i.e., the phospholipid). For example, the liposome composition can contain other lipids than the phospholipids or derivatives of the other lipids, membrane stabilizers, antioxidants and the like, as desired. The other lipids than the phospholipids can be lipids having a hydrophobic group such as a long chain alkyl group in the molecule thereof but not containing a phosphate group in the molecule thereof, and are not specifically restricted. Examples of the other lipids include glyceroglycolipids, sphingoglycolipids, sterol derivatives such as cholesterol, and their derivatives such as their hydrogenation products. Examples of the cholesterol derivatives include those sterols which have a cyclopentanohydrophenanthrene ring. For example, among these, cholesterol can be contained in an exemplary liposome composition. Examples of the antioxidants include ascorbic acid, uric acid, and tocopherol homologues, or vitamin E. Tocopherol includes four isomers, namely, α-, β-, γ- and δ-tocopherols, and any of such isomers can be used.

In an exemplary embodiment, the composition of the lipid bilayer of the liposome composition can be 100 to 50 mol % of phospholipid and 0 to 50 mol % of cholesterol, for example, 70 to 50 mol % of phospholipid and 30 to 50 mol % of cholesterol.

The liposome composition can include a first liposome having an outer membrane comprised of a multilayered lipid bilayer, and a plurality of second liposomes which are accommodated in a first liposome inner region defined by the outer membrane and each of which has an outer membrane comprised of a lipid bilayer. The lipid bilayer of the second liposomes can be multilayered. The multilayered bilayer of the first liposome includes multiple bilayers. For example, the multilayered bilayer of the second liposomes includes multiple bilayers. The liposome composition has second liposome inner regions each defined by the outer membrane of each of the second liposomes.

An exemplary liposome composition includes, as modes thereof, an empty liposome in which no drug is encapsulated, and a liposome in which a drug is encapsulated.

The outside diameter of the first liposome can be 1 to 20 μm, for example, 3 to 10 μm. Such an outside diameter can lead to excellent sustained release properties and can enable easy administration even through thin needles. In addition, the outside diameter of the second liposomes is not particularly limited. For example, the outside diameter of the second liposomes can be 100 to 800 nm, from a viewpoint of drug encapsulation amount and excellent sustained release properties.

In an exemplary embodiment, the plurality of second liposomes are present independently from each other in the first liposome, and the number of second liposomes is not particularly limited.

An exemplary liposome composition has an ion gradient formed at least between each of the second liposome inner regions and the outside of the first liposome. Hereinafter, the term “ion” refers to an ion forming the ion gradient. In an exemplary embodiment, that an ion gradient is formed between each of the second liposome inner regions and the outside of the first liposome can, for example, mean any of: (1) that a difference in ion concentration is present across the outer membrane of a second liposome, between the second liposome inner region and both of the first liposome inner region and the outside of the first liposome; (2) that a difference in ion concentration is present across the outer membrane of the first liposome, between the second liposome inner region as well as the first liposome inner region and the outside of the first liposome; and (3) that a difference in ion concentration is present across the outer membrane of the second liposome, between the second liposome inner region and the first liposome inner region and that a difference in ion concentration is present across the outer membrane of the first liposome, between the first liposome inner region and the outside of the first liposome (in this case, the ion concentration in the first liposome inner region is a value between the ion concentration in the second liposome inner region and the ion concentration in the outside of the first liposome).

In an exemplary embodiment, from a viewpoint of increasing the introduction of the drug, the ion concentration in the second liposome inner regions can be the highest. In addition, a setting can be made in which (the ion concentration in the second liposome inner regions)≧(the ion concentration in the first liposome inner region)>(the ion concentration in the outside of the first liposome). For example, a setting can be made in which (the ion concentration in the second liposome inner regions)>(the ion concentration in the first liposome inner region)≧(the ion concentration in the outside of the first liposome). For example, a setting can be made in which (the ion concentration in the second liposome inner regions)>(the ion concentration in the first liposome inner region)>(the ion concentration in the outside of the first liposome). For example, the case where (the ion concentration in the second liposome inner regions)=(the ion concentration in the first liposome inner region)=(the ion concentration in the outside of the first liposome) is excluded. Where proton gradient (pH gradient) is used as the ion gradient, a high ion concentration (proton concentration) corresponds to a low pH. For example, in this case, the pH in the second liposome inner regions is the lowest.

In an exemplary embodiment, before and after the introduction of a drug into empty liposomes, the shape and outside diameter of the first liposome as well as the shape and outside diameter of the second liposomes are substantially the same. In an exemplary embodiment, in the liposomes into which a drug has been introduced, the outside diameter of the first liposome and the outside diameter of the second liposomes are the same as those in the empty liposomes into which no drug has been introduced.

In the case where a drug is enveloped in the liposome composition, such a liposome composition can contain the drug in the second liposome inner regions or in the second liposome and first liposome inner regions.

The amount of the drug contained in the liposome composition is not particularly limited, and can be appropriately controlled according to the use of the composition. The amount of the drug, in terms of molar ratio [drug (mol)/total lipid (mol)] thereof based on the total lipid possessed by the liposome composition (the total amount of lipid(s) used in preparation of the liposome composition), can be not less than 0.05, and can be 0.06 to 0.14.

### Ion Gradient Method

The ion gradient method is a method in which an ion gradient is formed between the inside and the outside of a liposome membrane, and a drug added to the outside is transmitted through the liposome membrane according to the ion gradient, whereby the drug is encapsulated in the inside of the liposome. The ion gradient can be a proton gradient (e.g., a pH gradient). In the ion gradient method, empty liposomes in which no drug is encapsulated are prepared, and a drug is added to an outer liquid around the empty liposomes, whereby the drug can be introduced into the liposomes.

In an exemplary embodiment, provided is a liposome composition for encapsulating a drug by the ion gradient method, and a liposome composition in which a drug has been encapsulated by the ion gradient method. Among others, a pH gradient method in which pH gradient is used as the ion gradient can be applied.

As an exemplary method of forming a pH gradient, a liposome is formed by using an acidic-pH buffer (for example, a citric acid solution of pH 2 to 3) as a first inner aqueous phase and/or a second inner aqueous phase, and then the pH in the outside of the first liposome is controlled to within the vicinity of neutrality (for example, a buffer of pH 6.5 to 7.5), whereby a mode can be realized in which a pH gradient is formed such that the inside of the second liposomes and the inside of the first liposome are at lower pH whereas the outside of the first liposome is at a higher pH.

For example, a pH gradient can also be formed through an ammonium ion gradient. In this case, for example, a liposome is formed by using an ammonium sulfate solution as a first inner aqueous phase and/or a second inner aqueous phase, and then the ammonium sulfate in the outer aqueous phase for the first liposome is removed or diluted, whereby an ammonium ion gradient is formed at least between the inside of the second liposomes as well as the first liposome and the outside of the first liposome.

This can ensure that due to the ammonium ion gradient thus formed, outflow of ammonia from the inner aqueous phases in the first liposome and the second liposomes into the outer aqueous phase for the first liposome takes place. As a result, protons left by the ammonia are accumulated in the inner aqueous phases, whereby a pH gradient is formed, and the inner aqueous phases in the first liposome and the second liposomes become more acidic than the outer aqueous phase for the first liposome.

### Drug to be Encapsulated

As the drug to be encapsulated in the liposome composition, a drug can be used without any special restriction. The drug can be encapsulated into liposomes by the ion gradient method. Such a drug can be an ionizable amphipathic drug, for example, an amphipathic weakly basic drug. In addition, from a viewpoint of effect, the drug can be a drug for which sustained release properties in local administration are expected, for example, any of drugs for treatment of cerebral vascular disorder, Parkinson's disease, dementia, etc., analgesic agents, local anesthetics, and anti-malignancy agents. Examples of these drugs include donepezil, rivastigmine, galanthamine, physostigmine, heptylphysostigmine, phenserine, tolserine, symserine, thiatolserine, thiacymserine, neostigmine, huperzine, tacrine, metrifonate, minocycline, fasudil hydrochloride, nimodine, morphine, bupivacaine, ropivacaine, levobupivacaine, tramadol, lidocaine, and doxorubicin. Other examples include dopamine, L-DOPA, serotonin, epinephrine, codeine, meperidine, methadone, morphine, atropine, decyclomine, metixene, propantheline, imipramine, amitriptyline, doxepin, desipramine, quinidine, propranolol, chlorpromazine, promethazine, and perphenazine.

### Liposome First Inner Aqueous Phase Solution

In an exemplary process of producing the liposome composition, a first inner aqueous phase solution to be used in a step of preparing a first emulsion contains a compound for forming the ion gradient.

The ion for forming the ion gradient can be the proton, as above-mentioned. In addition, examples of the compound for forming the ion gradient (pH gradient) include those compounds which generate proton, ammonium ion or a protonated amino group through ionization. Examples of such a compound include: sulfates such as ammonium sulfate, dextran sulfate, and chondroitin sulfate; hydroxides; phosphoric acid, glucuronic acid, citric acid, carbonic acid, hydrogencarbonates, nitric acid, cyanic acid, acetic acid, benzoic acid, and their salts; halides such as bromides, and chlorides; inorganic or organic anions; and anionic polymers.

In the case where a weakly basic drug (for example, any of the above-mentioned ones) is encapsulated in the inner aqueous phase (at least the second inner aqueous phase) in the liposome composition according to an exemplary aspect by the pH gradient method, the drug is protonated by the protons present in the inner aqueous phase, to be thereby electrically charged. As a result, the drug is hampered from diffusing to the outside of the liposome, so that the drug is maintained in the liposome inner aqueous phase.

In the case where the compound for forming the ion gradient is ionized, anions such as sulfate ions are generated together with the ions (cations) for forming the ion gradient such as protons. In this case, if the anion forms a salt or complex with the protonated weakly basic drug, the drug can be maintained in the inner aqueous phase more stably. In other words, the compound for forming the ion gradient can be a compound which generates, through ionization, a counter ion (anion) for the basic drug and which is capable of forming a salt or complex with the basic drug. Such a counter ion is not specifically restricted so long as it is a pharmaceutically permissible anion. For example, the counter ion is a sulfate ion. As a compound for generating the sulfate ion, ammonium sulfate can be used, but the compound may also be selected from other compounds such as dextran sulfate and chondroitin sulfate. In addition, other examples of the counter ion include anions generated through ionization from hydroxides, phosphates, glucuronates, citrates, carbonates, hydrogencarbonates, nitrates, cyanates, acetates, benzoates, bromides, chlorides, and other inorganic or organic anions, or anionic polymers, etc.

In an exemplary embodiment, the concentration of the compound for forming the ion gradient in the first inner aqueous phase solution can be 50 to 500 mM, for example, 100 to 300 mM.

In an exemplary method of producing the liposome composition, the solvent to be used in preparation of the lipid-containing solution in the step of preparing the first emulsion is a water-miscible solvent. The water-miscible solvent means a solvent which dissolves the phospholipid(s) and other membrane component(s) used in the production of the liposome composition according to an exemplary aspect and which is miscible with water. Examples of the water-miscible solvent include ethanol, methanol, isopropyl alcohol, and butanol.

In an exemplary embodiment, solvents which are not miscible with water (referred to also as water-immiscible solvents; examples include water-immiscible organic solvents such as chloroform) are not used. For example, when a water-immiscible solvent is used in the step of preparing the first emulsion, the liposome obtained does not have a form in which a plurality of small liposomes and a first inner aqueous phase are contained in a large liposome; instead, the liposome obtained merely has a form such as a so-called multivesicular liposome (MVL) in which individual liposomes are simply gathered, like expanded polystyrene.

The amount of lipid(s) as a liposome raw material (the total amount of phospholipid(s) and other lipid(s)) can be 20 to 100 mass %, for example, 20 to 60 mass %, based on the water-miscible solvent.

In the first emulsion (the mixture of the lipid-containing water-miscible solvent and the ion-containing first inner aqueous phase solution), other component(s) than the components capable of constituting the lipid bilayer can fill up the inner regions of the second liposomes constituting the liposome composition of an exemplary aspect. Part of a second inner aqueous phase solution, which will be described later, may be additionally mixed in the inner regions of the second liposomes.

The method for preparing the first emulsion is not specifically restricted, and any suitable method can be used.

In the case where the pH gradient method is used, the pH of the inner aqueous phase (the first and/or second liposome inner region) can be controlled, as desired. For example, in the case where citric acid as a compound for forming an ion gradient is used in the first inner aqueous phase solution, a pH gradient between the inner aqueous phase (the second liposome inner regions) and the outer aqueous phase (the first liposome inner region and/or the outside of the first liposome) can be preliminarily formed. For example, in this case, the difference in pH between the inner aqueous phase and the outer aqueous phase is not less than three.

In the case where ammonium sulfate is used, a pH gradient is formed by chemical equilibrium, which can make it unnecessary to preliminarily control the pH of the inner aqueous phase solution. In this case, if the same solution as the outer aqueous phase is used as the second inner aqueous phase, formation of an ion gradient begins from the time of formation of the second emulsion, and a further gradient is formed by replacement of the outer liquid. In the case where the same ammonium sulfate solution as the first inner aqueous phase is used as the second inner aqueous phase, it is considered that an ion gradient is formed at the time of replacement of the outer liquid.

In the preparation of the liposome composition according to an exemplary aspect, the lipid-containing water-miscible solvent and the first inner aqueous phase solution to be added thereto can be used in a volume ratio (of the first inner aqueous phase solution to the water-miscible solvent) in a range from 0.7 to 2.5, for example, from 1.0 to 2.0.

### Liposome Second Inner Aqueous Phase Solution

In an exemplary embodiment, after the preparation of the first emulsion by adding the first inner aqueous phase solution to the lipid-containing water-miscible solvent, a step of adding the second inner aqueous phase solution to the first emulsion is conducted, in which the second inner aqueous phase solution is not specifically restricted. Examples of the second inner aqueous phase solution include the same solution as the first inner aqueous phase, a HEPES solution, a NaCl solution, and aqueous solutions of sugar such as glucose and sucrose. In an exemplary embodiment, the same solution as the first inner aqueous phase is employed. In an exemplary embodiment, the first inner aqueous phase and the second inner aqueous phase are each an aqueous ammonium sulfate solution. The first emulsion and the second inner aqueous phase solution to be added thereto can be used in a volume ratio of [the second inner aqueous phase solution] to [the first emulsion (=first inner aqueous phase solution+water-miscible solvent)] of not less than 0.7, for example, in a range of from 0.7 to 2.5, for example, in a range of from 1.0 to 1.5.

In the second emulsion, other component(s) than the component(s) capable of constituting the lipid bilayer can fill up the first liposome inner region (exclusive of the second liposomes) constituting the liposome composition of an exemplary aspect. The first liposome inner region (exclusive of the second liposomes) may contain part of the first emulsion.

The method for preparation of the second emulsion is not specifically restricted, and any suitable method can be used.

### Liposome Outer Aqueous Phase Solution

The process for producing the liposome composition according to an exemplary aspect includes a step of replacing the outer aqueous phase of the second emulsion with an aqueous solution which is lower than the first inner aqueous phase solution in the concentration of the compound for forming the ion gradient.

Where the outer aqueous phase of the first liposome after preparation of the second emulsion is changed by replacement of the liposome second inner aqueous phase solution or the mixed liquid containing the liposome first inner aqueous phase solution and the liposome second inner aqueous phase solution with an aqueous solution which is at least lower than the first inner aqueous phase solution in the concentration of the compound for forming the ion gradient, it is ensured that an ion gradient is formed at least between each of the second liposome inner regions and the outside of the first liposome, that the water-miscible solvent is removed from within the liposome composition system, and that the liposome obtained can be provided with the form possessed by the liposome composition according to an exemplary aspect.

As the outer aqueous phase for replacement that is used in an exemplary process for production of the liposome composition, an aqueous solution at least lower than the first inner aqueous phase solution in the concentration of the compound for forming the ion gradient is used. For example, a HEPES solution, a NaCl solution, or an aqueous solution of sugar such as glucose and sucrose is used. The pH of the outer aqueous phase can be adjusted by use of a buffer. Taking into account the decomposition of lipid and the pH gap at the time of administration into a living body, the pH can be controlled to within a range of pH 5.5 to 8.5, for example, a range of pH 6.5 to 7.5. Osmotic pressures of the inner aqueous phase and the outer aqueous phase for the liposome are not particularly limited. The osmotic pressures can be controlled to within such ranges that the liposome would not be broken by the difference between the osmotic pressures. In consideration of physical stability of the liposome, a smaller difference in osmotic pressure can be more desirable.

One exemplary embodiment of the outer aqueous phase for replacement is an aqueous solution that is lower than the first inner aqueous phase solution and the second inner aqueous phase solution in the concentration of the compound for forming the ion gradient.

The process for producing the liposome composition according to an exemplary aspect may further include a step of introducing a drug into the inside of the liposome composition by a driving force due to the ion gradient. In the step of introducing a drug into the inside of the liposome composition by the driving force due to the ion gradient, for example, the drug is dissolved in water or the like. The resulting drug solution is added to a liposome mixture obtained upon replacement of the liposome outer aqueous phase with the liposome outer aqueous phase solution, followed by blending the admixture. For example, the blended admixture is stirred with heating at or above a phase transition temperature of the liposome membrane, whereby a liposome in which the drug is encapsulated can be produced.

### Administering Method

The method for administering the liposome composition according to an exemplary aspect is not specifically restricted. For example, the liposome composition is administered non-perorally and locally. For instance, subcutaneous, intramuscular, intraperitoneal, intrathecal, extradural or intraventricular administration can be selected. The administering method can be appropriately selected according to the relevant symptom. As a specific method for administration, the liposome composition can be administered by use of a syringe or a spray-type device. In addition, the administration can be carried out through a catheter inserted in a living body, for example, in a body lumen, for instance, in a blood vessel.

### EXAMPLES

Exemplary aspects will be described in more detail below by showing Examples, but such exemplary aspects are not restricted to the Examples.

The concentration and particle diameter of each of drug-filled liposomes prepared in Examples were determined as follows.

Phospholipid Concentration (mg/mL): Phospholipid concentration in a liposome suspension that is quantified by high performance liquid chromatography or phospholipids determination.

Cholesterol Concentration (mg/mL): Cholesterol concentration in a liposome suspension that is quantified by high performance liquid chromatography.

Total Lipid Concentration (mol/L): Total mol concentration (mM) of lipid(s) as membrane component(s) that is calculated from the phospholipid concentration and the cholesterol concentration.

Drug Concentration (mg/mL): The liposome composition was diluted with RO water (reverse osmosis-purified water) so that the total lipid concentration of the preparation obtained above would be about 20 to 30 mg/mL. Then, the diluted liposome composition was further diluted with methanol by a factor of 20, and the liposome was disintegrated. For the resulting solution, absorbance at 315 nm was quantified by high performance liquid chromatography using a UV absorptiometer. The concentration of encapsulated donepezil hydrochloride is shown in drug amount (mg)/total preparation amount (mL).

Drug Support Amount (molar ratio of drug/total lipid): The concentration of donepezil hydrochloride encapsulated in the liposomes is shown in molar ratio of drug/total lipid, calculated from the ratio of the drug concentration to the total lipid concentration.

Concentration of Donepezil Hydrochloride in Plasma (mg/mL): Sampled plasma was treated, and, for a supernatant obtained finally by centrifugation, fluorescence at an excitation wavelength (Ex) of 322 nm and a detection wavelength

(Em) of 385 nm was quantified by high performance liquid chromatography using a fluorophotometer.

Particle Diameter (μm): Average particle diameter of the first liposome measured by a light scattering diffraction particle size distribution analyzer Beckman Coulter LS230.

The abbreviations and molecular weights of components used are set forth below.

HSPC: Hydrogenated soybean phosphatidylcholine (molecular weight 790, SPC3 produced by Lipoid GmbH)
SPC: Soybean Phosphatidylcholine (molecular weight 779, NOF Corporation)
DMPC: Dimyristoylphosphatidylcholine (molecular weight 677.9, NOF Corporation)
Chol: Cholesterol (molecular weight 388.66, produced by Solvay S.A.)
PEG5000-DSPE: Polyethylene glycol (molecular weight 5,000)-Phosphatidylethanolamine (molecular weight 6081, NOF Corporation)
Donepezil hydrochloride (molecular weight 415.95, UINAN CHENGHUI-SHUANFDA Chemical Co., Ltd.)

### Preparation of Different Inner Aqueous Phases

### Preparation Examples 1 to 4

### (1) Preparation of Empty Liposome

HSPC and cholesterol in respective amounts of 1.41 g and 0.59 g were weighed so that HSPC/Chol=54/46 (molar ratio, here and hereafter), then 4 mL of anhydrous ethanol was added thereto, and dissolution was effected by heating. To the ethanol solution of lipid thus obtained by dissolution, there was added 100 mM (Preparation Example 1), 150 mM (Preparation Example 2) or 250 mM (Preparation Example 3) of an aqueous ammonium sulfate solution or 300 mM of an aqueous citric acid solution (pH 3.0) (Preparation Example 4) heated to about 70° C. in the same amount (4 mL) as ethanol. Each admixture was heated and stirred for about ten minutes, to form an emulsion. Furthermore, the emulsion was admixed with 10 mL of 20 mM HEPES/0.9% sodium chloride (pH 7.5) heated to about 70° C., followed by heating and stirring for about ten minutes. After the heating was over, the liposomes were immediately cooled with ice.

### (2) Formation of pH Gradient

The liposomes obtained as above were dispersed in 20 mM HEPES/0.9% sodium chloride (pH 7.5) added thereto, followed by centrifugation at 3,500 rpm for 15 minutes, to precipitate the liposomes. Thereafter, the supernatant was removed, and subsequently the liposomes were dispersed in 20 mM HEPES/0.9% sodium chloride of pH 7.5 added thereto, followed by centrifugation in the same manner as above. This step was repeated three times, followed by re-dispersing in 20 mM HEPES/0.9% sodium chloride of pH 7.5, to form a pH gradient.

### (3) Introduction of Drug by pH Gradient

After the formation of the ion gradient, the amounts of HSPC and cholesterol of the liposomes were determined, and total lipid concentration was calculated. Based on the total lipid concentration thus calculated, the amount of donepezil hydrochloride (DNP, molecular weight 415.95) for realizing a DNP/total lipid (mol/mol) ratio of 0.16 was calculated. After the amount of DNP was weighed, a DNP solution (drug solution) of a concentration of 20 mg/mL was prepared by use of RO water. A predetermined amount of DNP solution preliminarily heated to 65° C. was added to the liposome solution heated to 65° C., followed by heating and stirring at 65° C. for 60 minutes, to effect introduction of the drug. After the introduction of the drug, the liposomes were immediately cooled with ice.

### (4) Removal of Unencapsulated Drug

After the introduction of the drug, the liposomes were dispersed in 20 mM HEPES/0.9% sodium chloride (pH 7.5) added thereto, followed by centrifugation at 3,500 rpm for 15 minutes, to precipitate the liposomes. Thereafter, the supernatant was removed, and subsequently the liposomes were dispersed in 20 mM HEPES/0.9% sodium chloride (pH 7.5) added thereto, followed by centrifugation in the same manner as above. This step was repeated three times, thereby removing the unencapsulated drug.

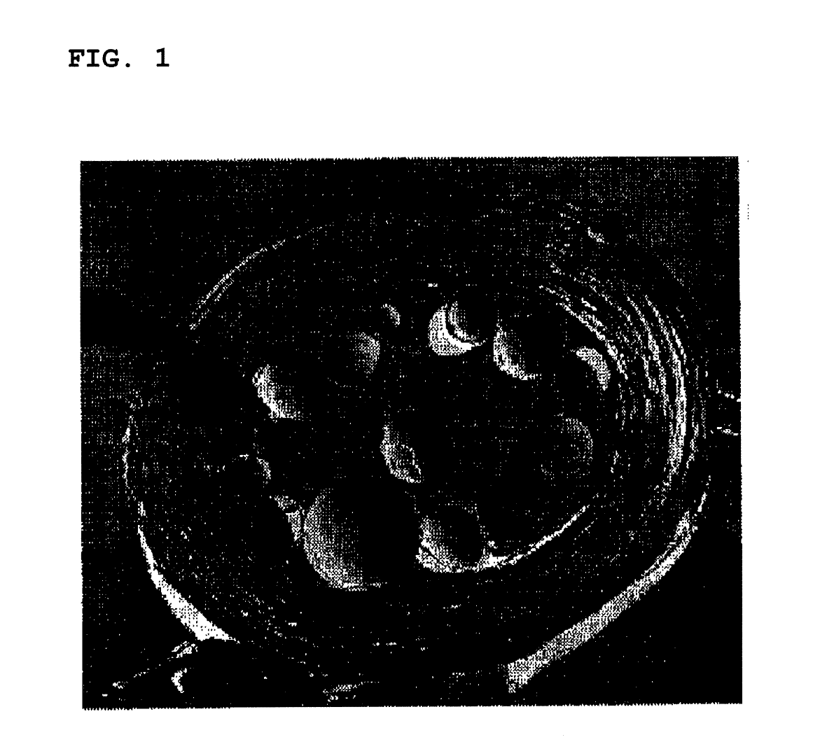

For the liposome compositions of Preparation Examples 1 to 4 obtained by the above-mentioned producing method, the first inner aqueous phases, membrane compositional ratios, drug support amounts (molar ratios of drug/total lipid) and particle diameters are set forth in Table 1. As a result of electron microscope observation, the liposome compositions according to exemplary aspects appeared as shown in FIG. 1, in which a plurality of vesicles (second liposomes) are present in each liposome (first liposome), and in which the outer membrane of each liposome is composed of a multilayered lipid bilayer. Moreover, notwithstanding that the liposome has the thick multilayered lipid bilayer and contains therein the plurality of vesicles each having the multilayered lipid bilayer in the same manner, a pH gradient sufficient for introduction of a drug can be formed between the inside and the outside of the liposomes after the formation of the liposomes. Consequently, the drug can be encapsulated highly efficiently, based on the pH gradient.

FIG. 1 is a photograph upon transmission electron microscope (TEM) observation of a section of the liposome after the drug introduction, produced in Preparation Example 2 according to this Example. The magnification is 32,000. The liposome shown in FIG. 1 is divided substantially at the center of the liposome. The liposome shown in FIG. 1 includes a first liposome having an outer membrane composed of a multilayered lipid bilayer, and a plurality of second liposomes which are accommodated in the first liposome inner region defined by the outer membrane and each of which has an outer membrane composed of a multilayered lipid bilayer. In FIG. 1, the outside diameter of the first liposome is about 4 μm, and the outside diameter of the second liposomes is 100 to 800 nm.

In addition, for the liposome compositions of Preparation Examples 1 to 4 obtained by the above-mentioned producing method, it has been made clear that the encapsulation amount of the drug is enhanced depending on an increase in the concentration of ammonium sulfate in the inner aqueous phase. While not wishing to be bound by any particular theory, this is considered to be because a drug holding capability was enhanced based on the amount of protons remaining in the inner aqueous phase. It is considered, therefore, that an ammonium sulfate concentration of not less than 150 mM is exemplary, in order to obtain a higher drug encapsulation amount. In the case where a citric acid solution of pH 3.0 was used as the inner aqueous phase in place of ammonium sulfate, a liposome having a high drug encapsulation amount was obtained in the same manner.

TABLE 1

| | First inner | Lipid | Volume | Volume | | Drug/Total | Average particle |
|---|---|---|---|---|---|---|---|
| | aqueous | composition | ratio, | ratio, | | lipid | diameter of first |
| | phase | (mol/mol) | I | II | Drug | (mol/mol) | liposome (μm) |
| Preparation Example 1 | 100 mM AS | HSPC/Chol = 54/46 | 1 | 1.25 | Donepezil hydrochloride | 0.06 | 3.5 |
| Preparation Example 2 | 150 mM AS | HSPC/Chol = 54/46 | 1 | 1.25 | Donepezil hydrochloride | 0.09 | 6.8 |
| Preparation Example 3 | 250 mM AS | HSPC/Chol = 54/46 | 1 | 1.25 | Donepezil hydrochloride | 0.11 | 7.9 |
| Preparation Example 4 | pH 3.0 | HSPC/Chol = 54/46 | 1 | 1.25 | Donepezil hydrochloride | 0.14 | 6.0 |
| | 300 mM CA | | | | | | |
| Preparation Example 5 | 150 mM AS | HSPC/Chol = 54/46 | 2 | 1.6 | Donepezil hydrochloride | 0.11 | 6.4 |
| Preparation Example 6 | 150 mM AS | DMPC/Chol = 54/46 | 1 | 1.25 | Donepezil hydrochloride | 0.11 | 4.7 |
| Preparation Example 7 | 150 mM AS | HSPC/Chol = 54/46 | 1 | 1 | Donepezil hydrochloride | 0.07 | 6.7 |
| Preparation Example 8 | 150 mM A3 | HSPC/Chol = 54/46 | 1 | 1.6 | Donepezil hydrochloride | 0.09 | 6.8 |
| Preparation Example 9 | 150 mM AS | HSPC/Chol = 54/46 | 1 | 2 | Donepezil hydrochloride | 0.09 | 5.5 |
| Preparation Example 10 | 150 mM AS | HSPC/Chol = 54/46 | 2 | 1 | Donepezil hydrochloride | 0.09 | 7.8 |
| Preparation Example 11 | 150 mM AS | HSPC/Chol = 54/46 | 2 | 2 | Donepezil hydrochloride | 0.09 | 7.3 |
| Preparation Example 12 | 150 mM AS | HSPC/Chol = 54/46 | 1 | 1.6 | Bupivacaine hydrochloride | 0.08 | 8.0 |
| Preparation Example 13 | 250 mM AS | HSPC/Chol = 54/46 | 1 | 1.6 | Bupivacaine hydrochloride | 0.11 | 8.1 |
| Preparation Example 14 | 150 mM AS | HSPC/Chol = 54/46 | 1 | 1.25 | Ropivacaine hydrochloride | 0.09 | 9.3 |
| Preparation Example 15 | 250 mM AS | HSPC/Chol = 54/46 | 1 | 1.25 | Tramadol hydrochloride | 0.09 | 9.1 |
| Comparative Example 5 | 150 mM AS | HSPC/Chol = 54/46 | 0.5 | 1.6 | Donepezil hydrochloride | 0.02 | 13.4 |
| Comparative Example 6 | 150 mM AS | HSPC/Chol = 54/46 | 9.0 | 0 | Donepezil hydrochloride | 0.07 | 8.1 |
| Comparative Example 7 | 150 mM AS | HSPC/Chol = 54/46 | 9.0 | 1.6 | Donepezil hydrochloride | 0.05 | 9.6 |

Volume ratio, I: Volume ratio of first inner aqueous phase solution/ethanol
Volume ratio, II: Volume ratio of second inner aqueous phase/(first inner aqueous phase + ethanol)
AS: ammonium sulfate
CA: citric acid

### Investigation of Different Inner Aqueous Phase Volumes

### Preparation Example 5

HSPC and cholesterol in respective amounts of 1.41 g and 0.59 g were weighed so that HSPC/Chol=54/46, and they were dissolved in 4 mL of an ethanol solution. After the dissolution, the ethanol solution of lipid was admixed with two-fold amount (8 mL) of a 150 mM aqueous ammonium sulfate solution, followed by heating and stirring for about ten minutes. Subsequently, 19 mL of a 150 mM aqueous ammonium sulfate solution was added thereto, and the resulting admixture was heated and stirred for about ten minutes. Thereafter, a pH gradient was formed and drug introduction and removal of the unencapsulated drug were conducted, in the same manner as in Preparation Examples 1 to 4. As a result, a high drug encapsulation amount was obtained in the same manner as in Preparation Examples 1 to 4, as shown in Table 1.

### Preparation Example 6

As a phospholipid, DMPC having a small alkyl group chain length was used. The DMPC and cholesterol in respective amounts of 2.70 g and 1.30 g were weighed so that DMPC/Chol=54/46, and were dissolved in 4 mL of an ethanol solution. Subsequently, 4 mL of a 150 mM aqueous ammonium sulfate solution was added to the ethanol solution of DMPC and cholesterol, followed by heating and stirring for about ten minutes. Thereafter, 10 mL of a 150 mM aqueous ammonium sulfate solution was added thereto, followed by heating and stirring for about ten minutes. Subsequently, a pH gradient was formed and drug introduction and removal of the unencapsulated drug were conducted, in the same manner as in Preparation Examples 1 to 4.

As a result, it was found out that, also in this case where the alkyl group chain length of the phospholipid was small, a high drug encapsulation amount was obtained in the same manner as in Preparation Examples 1 to 4, as shown in Table 1.

### Preparation of DNP Liposome by Other Method (Passive Method) than Ion Gradient Method

### Comparative Example 1

In drug introduction, the passive method (which is a comparative method) was used in place of the ion gradient method. The passive method is a method in which liposomes are prepared by preliminarily dissolving a drug in an inner aqueous phase. A predetermined amount of donepezil hydrochloride was preliminarily dissolved in physiological saline used as the first inner aqueous phase solution. Thereafter, liposome preparation was conducted in the same manner as in Preparation Examples 1 to 4. Physiological saline was used also as the outer aqueous phase.

As a result, it was made clear that encapsulation efficiency and the drug encapsulation amount are conspicuously lowered, as compared with the liposomes obtained by the exemplary method, as shown in Table 2. Furthermore, the liposome compositions according to Comparative Example 1 and the inventive examples were compared with each other as to drug release properties by use of an in-vitro evaluation system. As a result, it was made clear that the drug release was much faster in Comparative Example 1 than in the inventive examples.

From the foregoing, it was found clearly that in order to secure a drug encapsulation amount at a clinically sufficient level and to obtain long-term sustained release properties, it can be desirable to introduce a drug by the ion gradient method, particularly the pH gradient method. It was also made clear that where a drug is introduced by the ion gradient method, for example, the pH gradient method, the drug is protonated in the inside of the liposomes, and the liposomes have a layered structure as shown in FIG. 1, whereby longer-term sustained release properties can be obtained.

TABLE 2

| | | | Drug support | |
|---|---|---|---|---|
| | First inner | Lipid | amount Drug/ | Particle |
| Comparative | aqueous | composition | Total lipid | diameter |
| Example | phase solution | (mol/mol) | (mol/mol) | (μm) |
| 1 | Physiological | HSPC/ | 0.01 | 3.8 |
| | saline/ | Chol = | | |
| | Donepezil | 54/46 | | |
| | solution | | | |

### Preparation of Donepezil Liposome by Different Producing Methods

### Comparative Example 2

### Extrusion Method-1 (Particle Diameter: Around 300 Nm)

HSPC and cholesterol in respective amounts of 0.71 g and 0.29 g were weighed so that HSPC/Chol=54/46, and were dissolved with heating in 1 mL of anhydrous ethanol added thereto. The ethanol solution of lipid thus obtained, in an amount of 1 mL, was admixed with 9 mL of a 250 mM aqueous ammonium sulfate solution (inner aqueous phase) heated to about 70° C., followed by stirring by a ultrasonic device with heating, to prepare a crude liposome suspension. The crude liposome suspension thus obtained was passed sequentially through a filter (pore diameter 0.4 μm, Whatman plc; five times) attached to an extruder (The Extruder T.10, Lipexbiomembranes Inc.) heated to about 70° C., to prepare empty liposomes sized around 300 nm. Subsequently, while maintaining the liposomes in a heated state, an aqueous PEG5000-DSPE solution (37.7 mg/mL) was immediately added in such an amount as to be 0.75 mol % based on the total lipid, followed by heating and stirring, whereby membrane surfaces (outer surfaces) of the liposomes were modified with PEG. After the heating was over, the liposomes were immediately cooled with ice. The PEG-modified liposomes thus ice-cooled were subjected to outer liquid replacement by use of gel filtration replaced sufficiently with an outer aqueous phase solution (20 mM HEPES/0.9% sodium chloride solution (pH 7.5)). Thereafter, drug introduction was conducted so that drug/total lipid (mol/mol)=0.16. Subsequently, removal of the unencapsulated drug was conducted by use of gel filtration replaced sufficiently with 20 mM HEPES/0.9% sodium chloride solution (pH 7.5).

### Comparative Example 3

### Extrusion Method-2 (Particle Diameter: About 1 to 2 μm)

Preparation was conducted by use of an extruder in the same manner as in Comparative Example 2, except that a filter with a pore diameter of 2 μm was attached to the extruder, and the crude liposome suspension was passed through the filter five times, to obtain empty liposomes. The preparation was conducted by carrying out the drug introduction and removal of the unencapsulated drug in the same manner as in Comparative Example 2, to obtain multilamellar liposomes sized about 1 to 2 μm.

### Comparative Example 4

### Lipid Membrane Introduction Method

An aqueous citric acid hydrochloric acid solution of pH 6.5 as the first inner aqueous phase solution was added to an ethanol solution containing HSPC and donepezil dissolved therein, whereby donepezil hydrochloride was encapsulated in the lipid membrane. Donepezil liposomes were obtained in the same manner as in Comparative Example 1, except for the just-mentioned points.

For the donepezil liposomes obtained in Comparative Examples 2 to 4, the first inner aqueous phases, membrane compositional ratios, drug support amounts (molar ratios of drug/total lipid) and particle diameters are set forth in Table 3.

As a result, for the liposome compositions (Comparative Examples 2 and 3) with small particle diameters prepared by the extrusion method, high drug encapsulation amounts were obtained. On the other hand, for the preparation (Comparative Example 4) in which the drug was encapsulated in the lipid membrane, the drug/total lipid ratio was comparatively low, and the encapsulation efficiency was about 33%.

TABLE 3

| | | | Drug support | |
|---|---|---|---|---|
| Comparative | First inner | Lipid | amount Drug/ | Particle |
| Example | aqueous | composition | Total lipid | diameter |
| No. | phase solution | (mol/mol) | (mol/mol) | (μm) |
| 2 | 250 mM | HSPC/ | 0.13 | 0.29 |
| | ammonium | Chol = | | |
| | sulfate | 54/46 | | |
| 3 | 150 mM | | 0.15 | 1.7 |
| | ammonium | | | |
| | sulfate | | | |
| 4 | pH 6.5 citric | HSPC = 100 | 0.05 | 7.4 |
| | acid | | | |

### Donepezil Liposome Drug Dynamics 1

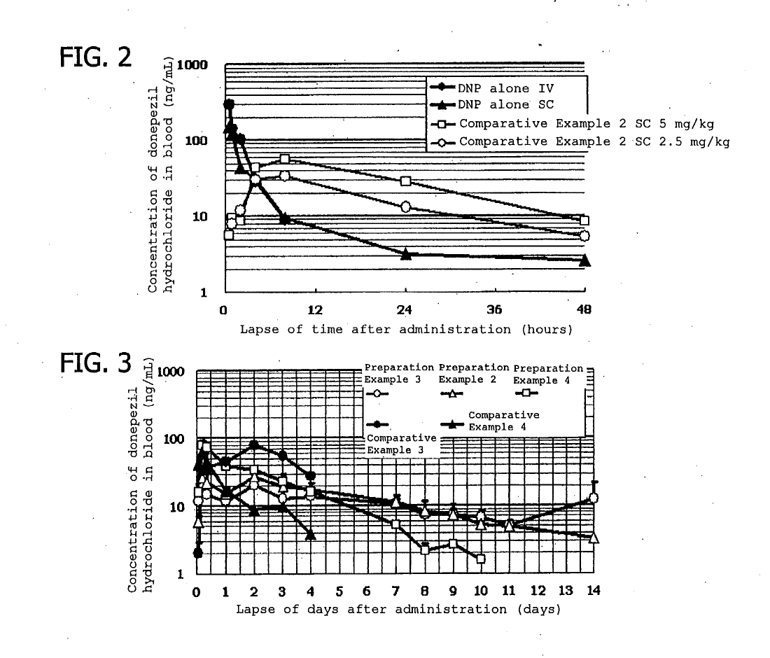

The donepezil liposome compositions prepared in Preparation Examples 2, 3 and 4 and Comparative Examples 2, 3 and 4 as well as donepezil used alone were subjected to a drug dynamics test. The donepezil liposome compositions in the volumes set forth in Table 4 as donepezil hydrochloride amount were each administered subcutaneously into a back part of a rat. Incidentally, for donepezil hydrochloride used alone, intravenous administration was conducted as well as the subcutaneous administration. After 1, 4, 8, 24, 48, 72, 96, 168, 192, 216, 240, 264, and 336 hours from the administration, blood was sampled from a tail vein, and subjected to centrifugation (6,000 rpm, ten minutes, at 4° C.), whereby plasma was obtained fractionally. The thus obtained plasma was treated, and the fluorescence intensity at an excitation wavelength (Ex) of 322 nm and a detection wavelength (Em) of 385 nm was determined by high performance liquid chromatography, thereby determining the concentration of donepezil hydrochloride in each plasma. The results are shown in FIGS. 2 and 3.

TABLE 4

| | | Results of |
|---|---|---|
| | | pharmacokinetics |
| Preparation | Dose | profile |
| Donepezil alone, intravenous | IV 2.5 mg/kg | FIG. 2 |
| administration | | |
| Donepezil alone, subcutaneous | SC 2.5 mg/kg | |
| administration | | |
| Comparative Example 2 | SC 2.5 mg/kg | |
| | SC 5 mg/kg | |
| Comparative Example 4 | SC 15 mg/kg | FIG. 3 |
| Preparation Example 2 | SC 25 mg/kg | |
| Preparation Example 3 | | |
| Preparation Example 4 | | |
| Comparative Example 3 | | |

IV = intravenous administration
SC = subcutaneous administration

As shown in FIG. 2, the concentration of donepezil hydrochloride in blood when donepezil hydrochloride used alone was administered intravenously or subcutaneously decreased rapidly after the administration, and the detection thereof continued only for eight hours and 48 hours after the administration, respectively. The liposome composition with a particle diameter of around 300 nm prepared in Comparative Example 2 did not show an initial burst, unlike donepezil used alone. Although it showed sustained release until 48 hours passed, its concentration already decreased below 10 ng/ml in 48 hours after the administration. While not wishing to be bound by any particular theory, it is believed that when the particle diameter is comparatively small as about 300 nm, the liposomes are liable to diffuse in the administration region, and donepezil hydrochloride is supposed to be transferred into lymph nodes or into blood together with the liposomes. It is therefore considered that the liposomes are lost early, and the expected sustained release properties cannot be obtained. In addition, in Comparative Example 4 in which the drug was encapsulated in the lipid membrane, the initial release amount is large, and thereafter the concentration of donepezil hydrochloride in blood was lowered rapidly, so that persistent sustained release properties could not be obtained. Probably, due to the high permeability of donepezil hydrochloride through the lipid membrane, the donepezil hydrochloride encapsulated in the membrane was not maintained stably, and, as a result, fast release properties were shown.

On the other hand, as shown in FIG. 3, the liposome compositions obtained in Preparation Examples 2, 3 and 4 did not show an initial burst, and showed a marked prolongation of sustained release time. Thus, sustained release properties over about two weeks could be obtained. As shown in FIG. 1, the liposome composition according to an inventive example contains a plurality of vesicles in each liposome, and the liposomes are covered with a thick lipid membrane having a layered structure composed of multiple layers. Due to these structures, permeability of the drug through the lipid membrane is considered to be suppressed. Further, it is considered that since the drug is maintained by the pH gradient method, release is restrained more, with the result that a remarkably long-term sustained release properties could be obtained. For example, where sulfate ions were present in the inner aqueous phase (Preparation Examples 2 and 3), the sustained release time was prolonged more. Thus, use ammonium sulfate as the inner aqueous phase solution is exemplary. This shows that an interaction of the protonated drug with the sulfate ions in the inner aqueous phase suppressed the release speed more, and, consequently, the long-term sustained release properties could be achieved.

From the foregoing, it was verified that in order to restrain the initial burst and achieve longer-term sustained release properties, it can be desirable that the liposomes have the form as shown in FIG. 1, the drug is encapsulated by the pH gradient method, and, for example, sulfate ions are present in the inner aqueous phase.

As for the liposome composition with a particle diameter of about 1.7 μm prepared by use of the extruder in Comparative Example 3, a high concentration in blood was maintained for four days, after which it was lowered rapidly.

### Donepezil Liposome Drug Dynamics 2

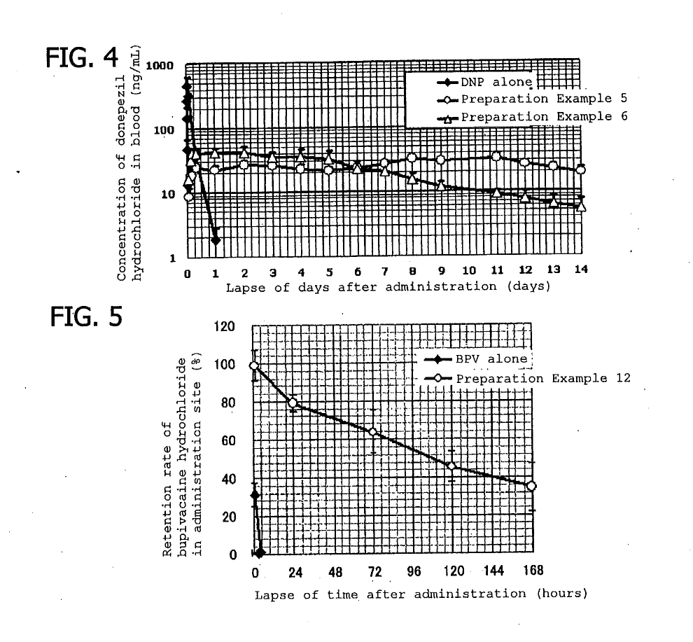

With the liposome composition according to an exemplary aspect, a high drug encapsulation amount can be obtained, so that the dose of the drug in subcutaneous administration can be enhanced. In view of this, the donepezil liposome compositions prepared in Preparation Examples 5 and 6 were administered subcutaneously into a back part of a rat in a donepezil hydrochloride dose of 50 mg/kg. Furthermore, for comparison, donepezil used alone was administered subcutaneously into a back part of a rat in a dose of 5 mg/kg. For the donepezil used alone, blood was sampled from a tail vein after lapses of 0.5, 1, 5, 10, 30, 120, 480, 1440, and 2880 minutes from the administration. For the liposome compositions, on the other hand, blood was sampled from a tail vein after lapses of 1, 3, 4, 8, 24, 48, 72, 96, 120, 144, 168, 192, 216, 264, 288, 312, and 336 hours from the administration. After the blood sampling, the same treatment as in <Donepezil Liposome Drug Dynamics 1> was conducted, and the concentration of donepezil hydrochloride in each plasma was determined. The results are shown in FIG. 4.

TABLE 5

| | | Results of |
|---|---|---|
| | | pharmacokinetics |
| Preparation | Dose | profile |
| Donepezil alone | SC 5 mg/kg | FIG. 4 |
| Preparation Example 5 | SC 50 mg/kg | |
| Preparation Example 6 | | |

As shown in FIG. 4, donepezil used alone showed its maximum concentration in blood after 0.5 hour from the administration, followed by a rapid lowering. After 48 hours, the concentration was already below the detection limit.

On the other hand, exemplary liposome compositions prepared in Preparation Examples 5 and 6 did not show an initial burst, and enabled an effective concentration at a clinically sufficient level over 14 days. For example, in the case of Preparation Example 5, an in-blood concentration of 20 to 30 ng/mL could be kept constantly for 14 days. Furthermore, it showed a trend that the sustained release properties would remain for more than 14 days. Thus, this liposome composition was verified to be a preparation that is excellent as a sustained release preparation.

### Investigation of First Inner Aqueous Phase/Ethanol Ratio and Second Inner Aqueous Phase/(First Inner Aqueous Phase+Ethanol) Ratio

### Preparation Examples 7 to 11 and Comparative Examples 5 to 7

HSPC and cholesterol in respective amounts of 1.41 g and 0.59 g were weighed so that HSPC/Chol=54/46, and were dissolved with heating in 4 mL of anhydrous ethanol added thereto. After the dissolution, the ethanol solution of lipid thus obtained was admixed with a 150 mM aqueous ammonium sulfate solution (first inner aqueous phase) heated to about 70° C., in each of the ratios shown in Table 1, followed by heating and stirring for about ten minutes. Subsequently, a second inner aqueous phase (20 mM HEPES/0.9% sodium chloride buffer (pH 7.5)) was added in each of the ratios shown in Table 1, based on the volume of (the first inner aqueous phase+ethanol), followed further by heating and stirring for about ten minutes. Thereafter, a pH gradient was formed and drug introduction and removal of the unencapsulated drug were carried out in the same manner as in Preparation Examples 1 and 2.

Table 1 shows the first inner aqueous phase/ethanol ratios, the second inner aqueous phase/(first inner aqueous phase+ethanol) ratios, the drug support amounts (molar ratios of drug/total lipid), and the particle diameters, for the liposome compositions prepared in Preparation Examples 7 to 11 and Comparative Examples 5 to 7.

Table 6 shows the comparison of Preparation Examples 2, 5, and 7 to 11 and Comparative Examples 5 to 7 as to drug support amount.

As a result, it was made clear that Preparation Examples 7 to 11 can yield a drug support amount comparable to those in Preparation Examples 2 and 5, and can yield a comparatively high drug encapsulation amount. In addition, the examples showed substantially the same behavior as to in-vitro release properties.

On the other hand, Comparative Example 5 gave a conspicuously low drug encapsulation amount. While not wishing to be bound by any particular theory, the reason is considered to reside in that due to the low first inner aqueous phase/ethanol ratio, the first emulsion was not formed cleanly, and, hence, something like lipid balls (aggregates of lipid) was formed. In addition, in regard of Comparative Examples 6 and 7, while not wishing to be bound by any particular theory, it is considered that since the first inner aqueous phase/ethanol ratio is high, something like a large liposome stable at this time point is formed, and the second inner aqueous phase is not liable to influence these structures. Further, as for in-vitro release properties in Comparative Examples 5, 6 and 7, there was a tendency toward a higher initial release speed, as compared with Preparation Examples 2, 5, and 7 to 11. While not wishing to be bound by any particular theory, from these results, it is supposed that in Comparative Examples 5, 6 and 7, the inner aqueous phase does not have a clearly formed structure, so that a sufficient amount of drug is not stably encapsulated in the inner aqueous phase, which leads to a slightly lowered drug encapsulation amount and a high initial release rate. While not wishing to be bound by any particular theory, in Comparative Example 3, the layers of the lipid membrane are considered to be very thin because of the structure in which a large inner aqueous phase is formed inside, though the liposome has a multilayered membrane. As a result, it is considered that the drug encapsulation amount is very high and the release is also very fast.

TABLE 8
Drug/Lipid (mol/mol)

| | | Volume ratio of second inner | | | | |
|---|---|---|---|---|---|---|
| | | aqueous phase/(first inner aqueous phase + EtOH) | | | | |
| | | 0 | 1/1 | 1.25/1 | 1.6/1 | 2/1 |
| Volume | 0.5/1 | | | | 0.02 | |
| ratio of | | | | | (Comparative | |
| first | | | | | Example 5) | |
| inner | 1/1 | | 0.07 | 0.09 | 0.09 | 0.09 |
| aqueous | | | (Preparation | (Preparation | (Preparation | (Preparation |
| phase/EtOH | | | Example 7) | Example 2) | Example 8) | Example 9) |
| | 2/1 | | 0.09 | | 0.11 | 0.09 |
| | | | (Preparation | | (Preparation | (Preparation |
| | | | Example 10) | | Example 5) | Example 11) |
| | 9/1 | 0.07 | | | 0.05 | |
| | | (Comparative | | | (Comparative | |
| | | Example 6) | | | Example 7) | |

### Preparation of Bupivacaine Hydrochloride Liposome according to an Inventive Example

### Preparation Examples 12 and 13

In the same manner as in Preparation Examples 1 to 4, HSPC and cholesterol were weighed in respective amounts of 4.23 g and 1.76 g so that HSPC/Chol=54/46, and were dissolved in 24 mL of an ethanol solution added thereto. After the dissolution, the ethanol solution of lipid was admixed with the same amount (24 mL) of a 150 mM or 250 mM aqueous ammonium sulfate solution, followed by heating with stirring for about ten minutes. Thereafter, 76.8 mL of a 150 mM or 250 mM aqueous ammonium sulfate solution was added, followed by heating with stirring for about ten minutes, and thereafter by immediate cooling with ice. Subsequently, centrifugation was conducted to replace the outer aqueous phase with 10 mM citric acid/0.9% sodium chloride of pH 6.5, thereby forming an ion gradient.

Thereafter, drug introduction was also conducted in the same manner as in Preparation Examples 1 to 4. Bupivacaine hydrochloride was used as the drug. After the amount of bupivacaine hydrochloride (BPV) was weighed, it was dissolved in RO water to prepare a BVP solution (drug solution) of a concentration of 10 mg/mL, which was stirred with heating at 65° C. for 60 minutes, whereby drug introduction was performed. After the drug introduction, the liposomes were immediately cooled with ice. Subsequently, removal of the unencapsulated drug was also conducted in the same manner as in Preparation Examples 1 to 4.

As a result, as shown in Table 1, it was made clear that in the case of using bupivacaine hydrochloride, it is possible to obtain a comparatively high drug encapsulation amount, like in the case of the donepezil liposome. From these results, it was verified that bupivacaine hydrochloride can also be introduced by the pH gradient method into the liposome having the structure of FIG. 1.

### Drug Dynamics in Bupivacaine Hydrochloride Liposome

For the bupivacaine hydrochloride liposome prepared in Preparation Example 12 and bupivacaine hydrochloride used alone, a drug dynamics test was conducted. Subcutaneous administration into a back part of a rat was conducted in a dose, in terms of the amount of bupivacaine hydrochloride, as set forth in Table 7. After lapses of 1, 24, 72, 120, and 168 hours from the administration of the bupivacaine hydrochloride liposome composition, and after lapses of 0.5, 4, and 24 hours from the administration of the bupivacaine hydrochloride used alone, back region subcutaneous tissue in the administration site was sampled and subjected to a homogenizing treatment. Subsequently, the homogenized solution was treated, the resultant sample solution was subjected to high performance liquid chromatography determination (UV-visible absorptiometer; measurement wavelength 210 nm), and the concentration of bupivacaine hydrochloride remaining in the back region subcutaneous tissue in the administration site was determined. The results are shown in FIG. 5. The retention rate of bupivacaine hydrochloride (used alone) in the administration site was lowered to below 1% in four hours after the administration. From this result, it was verified that bupivacaine hydrochloride used alone disappears from the administration site in several hours, which shows that a sustained in-blood concentration was not attained in such comparative example. On the other hand, the bupivacaine hydrochloride liposome gave a profile of sustained release from the administration site, and about 35% of bupivacaine hydrochloride remained on the seventh day from the administration. From these results, it was suggested that the liposome which was administered releases bupivacaine hydrochloride in the administration site in a sustained manner. From the foregoing, it is verified that the bupivacaine hydrochloride liposome obtained according to inventive examples has a long-term sustained release ability of not less than one week.

TABLE 7

| | | Results of |
|---|---|---|
| | | pharmacokinetics |
| Preparation | Dose | profile |
| Bupivacaine hydrochloride alone, | SC 5 mg/kg | FIG. 5 |
| subcutaneous administration | | |
| Preparation Example 12 | SC 5 mg/kg | |

### Preparation of Ropivacaine Hydrochloride Liposome According to an Inventive Example

### Preparation Example 14

A liposome composition was produced in the same manner as in Preparation Example 2, except that ropivacaine hydrochloride was used as the drug, to obtain a ropivacaine hydrochloride liposome.

As a result, as shown in Table 1, it was made clear that also in the case of using ropivacaine hydrochloride, it is possible to introduce the drug by the pH gradient method and to obtain a liposome composition having a comparatively high drug encapsulation amount, in the same manner as above. Also, as to in-vitro release properties, there was exhibited a release profile comparable to those in the cases of the donepezil hydrochloride liposome and the bupivacaine hydrochloride liposome having shown sustained release properties. This suggests that the use of ropivacaine hydrochloride provides release performance in the same manner as in the cases of donepezil hydrochloride and bupivacaine hydrochloride.

### Preparation of Tramadol Hydrochloride Liposome according to an Inventive Example

### Preparation Example 15

A liposome composition was produced in the same manner as in Preparation Example 3, except that tramadol hydrochloride was used as the drug, to obtain a tramadol hydrochloride liposome.

As a result, as shown in Table 1, it was verified that also in the case of using tramadol hydrochloride, it is possible to introduce the drug by the pH gradient method and to obtain a liposome composition having a comparatively high drug encapsulation amount, in the same manner as above.

What is claimed is:

1. A liposome composition, comprising:
a first liposome having an outer membrane comprised of a multilayered lipid bilayer; and
a plurality of second liposomes accommodated in a first liposome inner region defined by the outer membrane of the first liposome, the second liposomes each having an outer membrane comprised of a lipid bilayer,
wherein the liposome composition has second liposome inner regions each defined by the outer membrane of each of the second liposomes, and
an ion gradient is formed at least between each of the second liposome inner regions and the outside of the first liposome.

2. The liposome composition according to claim 1, wherein the ion gradient is a proton concentration gradient, and a pH in the second liposome inner region or a pH in the second liposome inner region and the first liposome inner region is lower than a pH in the outside of the first liposome.

3. The liposome composition according to claim 1, wherein the first liposome has an average particle diameter within a range of 1 to 20 μm.

4. The liposome composition according to claim 1, wherein a drug is contained in the second liposome inner region or in the second liposome and first liposome inner regions.

5. The liposome composition according to claim 4, wherein the drug is contained in a molar ratio (mol/mol) of not less than 0.05, based on total lipid.

6. A process for producing a liposome composition provided with an ion gradient between the inside and the outside of an outer membrane, the process comprising:
mixing a first inner aqueous phase solution containing a compound for forming the ion gradient with a lipid-containing water-miscible solvent in a volume ratio from 0.7 to 2.5 so as to prepare a first emulsion;
mixing a second inner aqueous phase solution with the first emulsion in a volume ratio of not less than 0.7 so as to prepare a second emulsion; and
replacing an outer aqueous phase of the second emulsion with an aqueous solution which is lower than the first inner aqueous phase solution in the concentration of the compound for forming the ion gradient.

7. The process for producing a liposome composition according to claim 6, wherein the ion gradient is a proton concentration gradient.

8. The process for producing a liposome composition according to claim 6, further comprising
a step of introducing a drug into the inside of the liposome composition by a driving force due to the ion gradient.

9. The liposome composition according to claim 2, wherein the first liposome has an average particle diameter within a range of 1 to 20 μm.

10. The liposome composition according to claim 2, wherein a drug is contained in the second liposome inner region or in the second liposome and first liposome inner regions.

11. The liposome composition according to claim 3, wherein a drug is contained in the second liposome inner region or in the second liposome and first liposome inner regions.

12. The process for producing a liposome composition according to claim 7, further comprising
a step of introducing a drug into the inside of the liposome composition by a driving force due to the ion gradient.

13. The liposome composition according to claim 1, wherein the outside of the first liposome includes an aqueous phase having a pH of 6.5 to 7.5.

14. The liposome composition according to claim 1, wherein the outside diameter of the second liposomes is 100 to 800 nm.

15. The liposome composition according to claim 1, wherein the lipid bilayer includes 100 to 50 mol % of phospholipid and 0 to 50 mol % of cholesterol.

16. The liposome composition according to claim 1, wherein the lipid bilayer includes phosphatidylcholine, phosphatidylglycerol, phosphatidic acid, phosphatidylethanolamine, phosphatidylserine, phosphatidylinositol, a sphingophospholipid, a natural or synthetic diphosphatidylphospholipid, a hydrogenation product of a phospholipid, or a combination thereof.

17. The process for producing a liposome composition according to claim 7, wherein the ion gradient is an ammonium ion gradient.

18. The process for producing a liposome composition according to claim 17,
wherein ammonia from the first and second inner aqueous phase solutions flows into the aqueous solution which is lower than the first inner aqueous phase solution in the concentration of the compound for forming the ion gradient, and
wherein protons left by the ammonia are accumulated in the first and second inner aqueous phase solutions, thus forming a pH gradient.

19. The process for producing a liposome composition according to claim 6, wherein the first inner aqueous phase solution is an ammonium sulfate solution, and the second inner aqueous phase solution is an ammonium sulfate solution.

20. The process for producing a liposome composition according to claim 6, wherein the lipid-containing water-miscible solvent is free from a water-immiscible solvent.

21. The liposome composition according to claim 1, wherein the lipid bilayer of the second liposomes is a multilayered lipid bilayer.
